(19) United States
(12) Patent Application Publication
(10) Pub. No.: US 2012/0258529 A1
(43) Pub. Date: Oct. 11, 2012

(54) APPARATUS FOR SEPARATING TARGET MOLECULES AND METHOD OF SEPARATING TARGET MOLECULES BY USING THE SAME

(75) Inventors: Hee-kyun LIM, Hwaseong-si (KR); Joon-ho KIM, Seongnam-si (KR); Myung-gwon LEE, Seoul (KR); Je-kyun PARK, Daejeon (KR)

(73) Assignees: KOREA ADVANCED INSTITUTE OF SCIENCE AND TECHNOLOGY, Daejeon (KR); SAMSUNG ELECTRONICS CO., LTD., Suwon-si (KR)

(21) Appl. No.: 13/272,338

(22) Filed: Oct. 13, 2011

(30) Foreign Application Priority Data
Apr. 5, 2011 (KR) 10-2011-0031285

(51) Int. Cl. C12N 5/078; C12M 1/12

(52) U.S. Cl. 435/325; 435/283.1

(57) ABSTRACT

An apparatus for separating target molecules includes a plurality of protruding portions on a first sidewall of a fluid channel to control a flow of a fluid containing the target molecules, and a fluid channel portion having a variable height for separating the target molecules depending on sizes of the target molecules.

### CROSS-REFERENCE TO RELATED APPLICATIONS

This application claims priority to Korean Patent Application No. 10-2011-0031285, filed on Apr. 5, 2011, and all the benefits accruing therefrom under 35 U.S.C. §119, the disclosure of which is incorporated herein in its entirety by reference.

### BACKGROUND

1. Field

Provided is an apparatus for separating target molecules and a method of separating target molecules by using the apparatus, and more particularly, an apparatus for separating target molecules which is capable of adjusting the cut-off size for the molecules and a method of separating target molecules by using the apparatus.

2. Description of the Related Art

A micro total analysis system (“μ-TAS”) is referred to as an integrated and small analysis system which analyzes biological samples through the pretreatment of the samples and provides analysis results. The number of target substances to be analyzed in drug development and screening is rising with the development of life sciences. At the same time, the demands for expensive reagents and samples are on the rise along with the need for cost reduction through ultra-micro quantity analysis. Emerging accordingly is the lab-on-a-chip technology for integrating the system in a single chip. In order to realize the technology, glass, silicone or plastic microchannels several to dozens of micrometers in size are formed by photolithography or micromachining, which are two widespread semiconductor manufacturing techniques. Then, fluids flowing in the microchannels are controlled microfluidically, i.e., by using the flow characteristics of the fluids.

### SUMMARY

Provided is an apparatus for separating target molecules which includes: a substrate including an elastomer, a fluid inlet portion on the substrate and including at least one fluid inlet passage into which a fluid containing at least one type of target molecules is injected; a fluid channel portion in fluid connection with the fluid inlet portion, and including a plurality of protruding portions on a first sidewall thereof to control a flow of the fluid, and a fluid outlet portion in fluid connection with the fluid channel portion, and including at least one fluid outlet passage through which the separated target molecules are discharged. A height of the fluid channel portion is variable so that the target molecules are separated depending on a size of the target molecules.

The apparatus for separating target molecules may further include a pressure member which overlaps the fluid channel portion and applies a pressure to the fluid channel portion so that the height of the fluid channel portion varies.

A ratio of the size of the target molecule to the height of the fluid channel portion may be from about 0.05 to about 0.3, and the target molecules may be separated toward the first sidewall.

The fluid channel portion may further include a plurality of first channel portions separated from each other by the plurality of protruding portions, and a plurality of second channel portions which alternate with the plurality of first channel portions, the plurality of second channel portions having a width smaller than a width of the plurality of first channel portions.

The width of the plurality of second channel portions may be larger than a height of the plurality of second channel portions.

A ratio of the size of the target molecule to the height of the plurality of second channel portions may be from about 0.05 to about 0.3, and the target molecules may be separated toward the first sidewall.

A method of separating target molecules may include: injecting a fluid containing at least one type of target molecules into a fluid inlet portion of an apparatus; separating the target molecules depending on a size of the target molecules by controlling a flow of the fluid with a fluid channel portion of the apparatus, which is in fluid connection with the fluid inlet portion; and discharging the fluid containing the separated target molecules through a fluid outlet portion of the apparatus, which is in fluid connection with the fluid channel portion. The apparatus includes a substrate including an elastomer, the fluid inlet portion on the substrate and including at least one fluid inlet passage, the fluid channel portion on the substrate and including a plurality of protruding portions on a first sidewall thereof and a variable height, and the fluid outlet portion on the substrate and including at least one fluid outlet passage.

The method may further include applying a pressure to the fluid channel portion so that the height of the fluid channel portion varies.

The height of the fluid channel portion may vary so that a ratio of the size of the separated target molecule to the height of the fluid channel portion is from about 0.05 to about 0.3.

The fluid channel portion may further include a plurality of first channel portions separated from each other by the plurality of protruding portions, and a plurality of second channel portions which alternate with the plurality of first channel portions, the plurality of second channel portions having a width smaller than a width of the plurality of first channel portions.

The width of the plurality of second channel portions may be larger than a height of the plurality of second channel portions.

The height of the plurality of second channel portions may vary so that a ratio of the size of the separated target molecule to the height of the plurality of second channel portions is from about 0.05 to about 0.3.

### BRIEF DESCRIPTION OF THE DRAWINGS

These and/or other aspects will become apparent and more readily appreciated from the following description of the embodiments, taken in conjunction with the accompanying drawings of which:

FIG. 1A is a perspective view illustrating an apparatus for separating target molecules according to an embodiment, and FIG. 1B is a plan view illustrating the apparatus of FIG. 1A;

FIGS. 2A to 2C are cross-sectional views illustrating second channel portions of an apparatus according to a comparison example;

FIGS. 3A to 3C are cross-sectional views illustrating second channel portions of an apparatus according to an embodiment;

FIG. 4A is a cross-sectional view illustrating a state when no pressure is applied to a fluid channel portion of an apparatus according to an embodiment, and FIG. 4B is a cross-sectional view illustrating a state when a pressure is applied to the fluid channel portion of FIG. 4A;

FIGS. 5A and 5D are graphs illustrating how red blood cells are separated by an apparatus according to an embodiment depending on a height of a fluid channel portion; and

FIGS. 6A and 6D are graphs illustrating how blood plasmas are separated by an apparatus according to an embodiment depending on a height of a fluid channel portion.

### DETAILED DESCRIPTION

Various example embodiments will now be described more fully with reference to the accompanying drawings in which some example embodiments are shown.

Detailed illustrative example embodiments are disclosed herein. However, specific structural and functional details disclosed herein are merely representative for purposes of describing example embodiments. This invention may, however, may be embodied in many alternate forms and should not be construed as limited to only the example embodiments set forth herein.

Accordingly, while example embodiments are capable of various modifications and alternative forms, embodiments thereof are shown by way of example in the drawings and will herein be described in detail. It should be understood, however, that there is no intent to limit example embodiments to the particular forms disclosed, but on the contrary, example embodiments are to cover all modifications, equivalents, and alternatives falling within the scope of the invention.

It will be understood that, although the terms ‘first’, ‘second’, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items.

It will be understood that when an element or layer is referred to as being “on” or “formed on,” another element or layer, it can be directly or indirectly on or formed on the other element or layer. That is, for example, intervening elements or layers may be present. In contrast, when an element or layer is referred to as being “directly on” or “directly formed on,” to another element, there are no intervening elements or layers present. Other words used to describe the relationship between elements or layers should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

In the drawings, the thicknesses of layers and regions are exaggerated for clarity.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

Reference will now be made in detail to embodiments, examples of which are illustrated in the accompanying drawings, wherein like reference numerals refer to the like elements throughout the description of the figures. In this regard, the present embodiments may have different forms and should not be construed as being limited to the descriptions set forth herein. Accordingly, the embodiments are merely described below, by referring to the figures, to explain aspects of the present description.

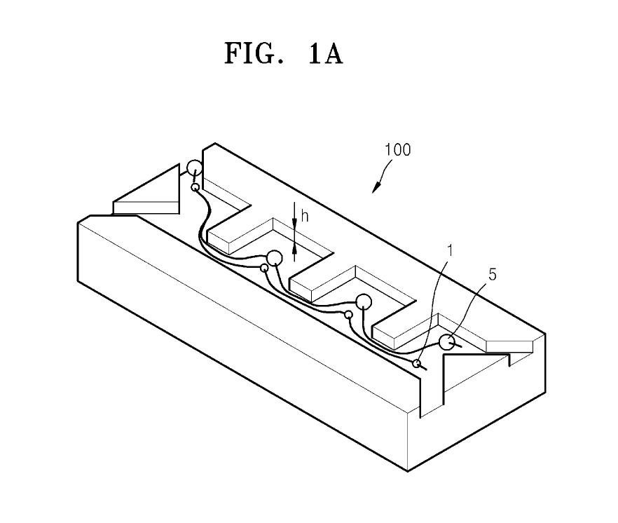

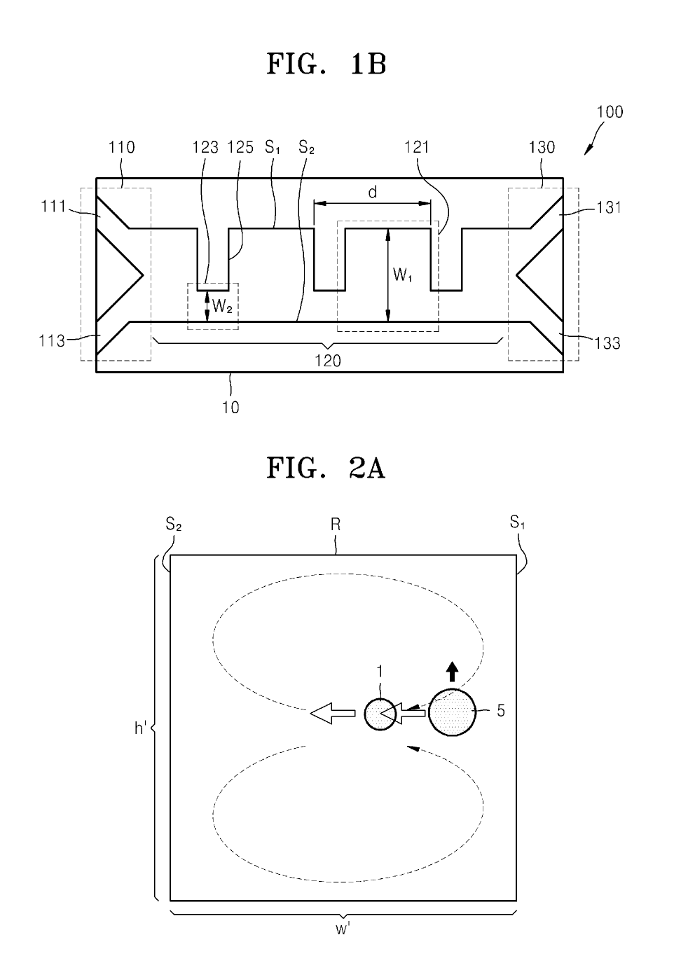

FIG. 1A is a perspective view illustrating an apparatus 100 for separating target molecules according to an embodiment, and FIG. 1B is a plan view illustrating the apparatus 100 for separating target molecules.

Referring to FIGS. 1A and 1B, the apparatus 100 for separating target molecules may include a fluid inlet portion 110 on a substrate 10, a fluid channel portion 120 in fluid connection with the fluid inlet portion 110 and having a plurality of protruding portions 125 on a first sidewall S₁ thereof, and a fluid outlet portion 130 in fluid connection with the fluid channel portion 120.

The substrate 10 may include an elastomer such as rubber, a silicone resin, or a polymer. The substrate 10 may be elastically deformable. In an embodiment, the fluid inlet portion 110, the fluid channel portion 120, and the fluid outlet portion 130 may be formed on a surface of the substrate 10 by photolithography, an etching process, or the like. A height h of the fluid channel portion 120 may elastically vary depending on a pressure applied to the fluid channel portion 120.

The fluid inlet portion 110 may be defined by an upper substrate (not illustrated) covering a groove which extends into the surface of the substrate 10, and the substrate 10. The fluid inlet portion 110 may include at least one fluid inlet passage. A fluid containing at least one type of target molecules may be injected into the fluid inlet portion 110. As illustrated in FIG. 1, the fluid inlet portion 110 may include a first fluid inlet passage 111 and a second fluid inlet passage 113. The first and second fluid inlet passages 111 and 113 may be separated from each other at a distance away from an entrance of the fluid channel portion 120, and join with each other at the entrance of the fluid channel portion 120. The first fluid inlet passage 111 may end at the first sidewall S₁ of the fluid channel portion 120, and the second fluid inlet passage 113 may end at a second sidewall S₂ of the fluid channel portion 120.

The fluid containing at least one type of target molecules may be injected into the first fluid inlet passage 111 of the fluid inlet portion 110. A size of a target molecule may be from about several nanometers to about dozens of micrometers. In one embodiment, for example, the size of the target molecule may be between about 10 nanometers and about 50 micrometers.

FIG. 1A illustrates a case when a fluid containing a first target molecule 1, and a second target molecule 5 larger than the first target molecule 1, is injected into the first fluid inlet passage 111. A buffer solution may be injected into the second fluid inlet passage 113. The buffer solution may adjust a flow rate of the fluid and a cut-off size for the target molecules. In one embodiment, for example, blood containing red blood cells and blood plasmas may be injected into the first fluid inlet passage 111, and a phosphate buffered saline (“PBS”) solution may be injected into the second fluid inlet passage 113. The apparatus 100 for separating target molecules may separate certain blood cells such as white blood cells, circulating tumor cells (“CTCs”), and hematopoietic stem cells (“HSCs”) from other cells.

Alternatively, the fluid containing the first and second modules 1 and 5 may be injected into the second fluid inlet passage 113, and the buffer solution may be injected into the first fluid inlet passage 111.

The fluid channel portion 120 may be defined by the upper substrate covering the groove in the surface of the substrate 10 and leading from the fluid inlet portion 110. Or, the substrate 10 may be on another substrate (not illustrated) such as a slide glass which is on an upper side of the substrate 10, where the groove is below the another substrate. In other words, the groove in the substrate 10 may be covered by the another substrate so that the fluid channel portion 120 is defined between the another substrate and a lower surface of the groove.

The fluid channel portion 120 may be a single, unitary, indivisible flow passage through which the fluid flows from the fluid inlet portion 110 toward the fluid outlet portion 130. The fluid channel portion 120 may include the plurality of protruding portions 125 on the first sidewall S₁ of the fluid channel portion 120. The plurality of protruding portions 125 may form a uneven pattern of the flow passage, and control the fluid such that the target molecules of different sizes contained in the fluid are separated depending on the sizes.

The plurality of protruding portions 125 may divide the fluid channel portion 120 into a plurality of first channel portions 121, and a plurality of second channel portions 123 that alternate with the plurality of first channel portions 121 in a flow direction of the fluid channel portion 120. In other words, the fluid channel portion 120 may include the plurality of first and second channel portions 121 and 123 which alternate with each other. A width W₂ of a second channel portion 123 may be smaller than a width W₁ of a first channel portion 121. Thus, a flow area of the second channel portion 123 may be smaller than a flow area of the first channel portion 121.

Although the protruding portion 125 has a rectangular cross-sectional shape in the plan view of FIG. 1 B, the shape is not limited thereto. The protruding portion 125 may have various shapes such as a serrated or semicircular shape. A distance d from a leading edge of one of the protruding portions 125 to a leading edge of another (e.g., adjacent) of the protruding portions 125 may be from about 100 micrometers to about 900 micrometers. In one embodiment, for example, the distance d may be about 700 micrometers. The width W₁ of the first channel portion 121 may be between about 200 micrometers and about 500 micrometers. In addition, the width W₂ of the second channel portion 123 may be between about 10 micrometers and about 100 micrometers. In one embodiment, for example, the width W₁ of the first channel portion 121 may be about 350 micrometers, and the width W₂ of the second channel portion 123 may be about 50 micrometers.

The height h of the fluid channel portion 120 may vary so that the target molecules of different sizes contained in the fluid are separated depending on the sizes. Here, the height h of the fluid channel portion 120 is referred to as a length of a side of the fluid channel portion 120 perpendicular to the widths W₁ and W₂ of the fluid channel portion 120. The height h of the fluid channel portion 120 is determined depending on the cut-off size for the target molecules to be separated when the apparatus 100 for separating target molecules is made or used. When the target molecules contained in the fluid pass through the fluid channel portion 120, the target molecules may be affected by inertial lift force and Dean drag force caused by a Dean flow. The inertial lift force is caused by a shear flow at a center and inner walls of the fluid channel portion 120. The Dean flow is referred to as a flow of the fluid across another flow of the fluid from the fluid inlet portion 110 toward the fluid outlet portion 130. The Dean flow is caused by a difference between the flow areas of the first and second channel portions 121 and 123 when the fluid passes through the first and second channel portions 121 and 123. The height h of the fluid channel portion 120 may be from about 10 micrometers to about 100 micrometers. In one embodiment, for example, the height h of the fluid channel portion 120 may be about 20 micrometers.

The Dean drag force and the inertial lift force affecting the molecules may vary depending on the size of a target molecule and the height h of the fluid channel portion 120. Therefore, the target molecules may be moved in different directions due to either one of the size of the target molecule or the height h of the fluid channel portion 120. The apparatus 100 for separating target molecules separates the target molecules without applying any external force such as an electric or magnetic field. Therefore, the apparatus 100 for separating target molecules preserves the characteristics of the molecules, and reduces or effectively prevents damage to the molecules.

The cut-off size for the target molecules to be separated may depend on the height h of the fluid channel portion 120. The shorter the height h of the fluid channel portion 120, the smaller the cut-off size for the target molecules to be separated may be. In other words, the shorter the height h of the fluid channel portion 120, the smaller the size of a target molecule may be which moves toward the first sidewall S₁ of the fluid channel portion 120 and be separated. In the apparatus 100 for separating target molecules, a ratio a/h of the size a for a target molecule to be separated, to the height h of the fluid channel portion 120, may be between about 0.05 and about 0.3. In one embodiment, for example, in a case when the size for the target molecule to be separated is about 4 micrometers, and the height h of the fluid channel portion 120 is about 20 micrometers, the ratio a/h may be about 0.2. Therefore, in the apparatus 100 for separating target molecules, the cut-off size for the target molecules to be separated may be changed by varying the height h of the fluid channel portion 120. The height h of the fluid channel portion 120, a height of the first channel portion 121, and a height of the second channel portion 123 may be identical to one another.

The fluid outlet portion 130 may be defined by the upper substrate covering the groove in the surface of the substrate 10 and leading from the fluid channel portion 120. The fluid outlet portion 130 may include at least one fluid outlet passage through which the separated target molecules are discharged. The fluid outlet portion 130 may include a first fluid outlet passage 131 and a second fluid outlet passage 133. The first fluid outlet passage 131 may effectively begin at the sidewall S₁ of the fluid channel portion 120. The second fluid outlet passage 133 may be separated from the first fluid outlet passage 131 and begin at the second sidewall S₂ of the fluid channel portion 120. The first and second fluid inlet passages 111 and 113, the first and second fluid outlet passages 131 and 133, and the first and second channel portions 121 and 123 may collectively form a single, unitary, indivisible flow passage.

The fluid containing at least one type of separated target molecules may be divided and discharged through the first and second fluid outlet passages 131 and 133, respectively. As one example embodiment, the fluid containing the second target molecule 5 may be discharged through the first fluid outlet passage 131, and the fluid containing the first target molecule 1 smaller than the second target molecule 5 may be discharged through the second fluid outlet passage 133. As another example embodiment, the fluid containing the red blood cells may be discharged through the first fluid outlet passage 131, and the fluid containing the blood plasmas may be discharged through the second fluid outlet passage 133.

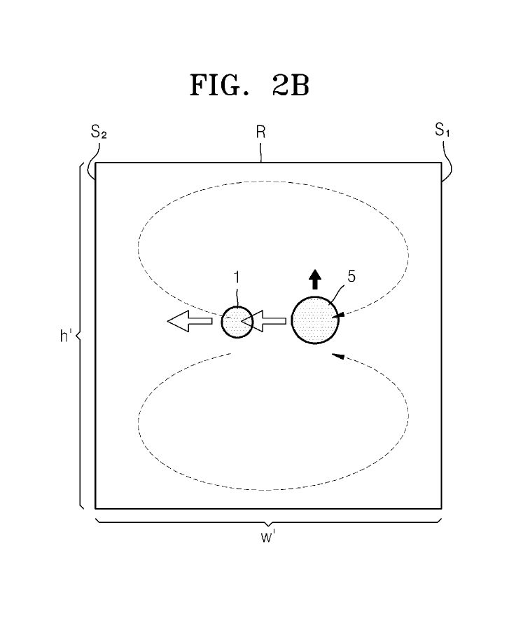

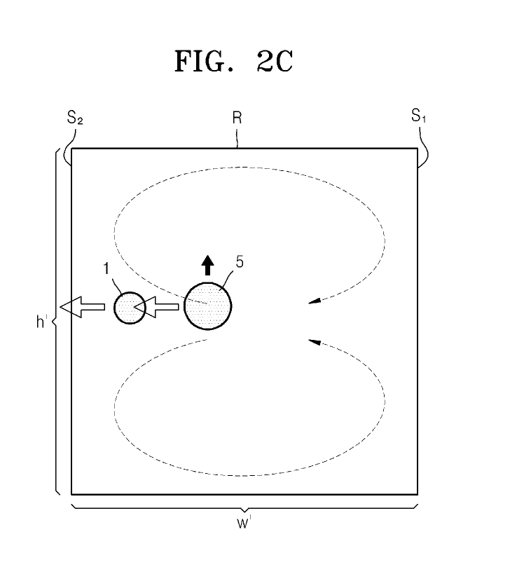

FIGS. 2A to 2C are cross-sectional views illustrating second channel portions of an apparatus according to a comparison example. FIG. 2A illustrates the second channel portion closest to the fluid inlet portion, and FIG. 2C illustrates the second channel portion farthest from the fluid inlet portion. FIG. 2B illustrates the second channel portion disposed between the second channel portions of FIGS. 2A and 2C. The widths w′ and the heights h′ of the second channel portions of the comparison example may be identical to one another. Here, the widths w′ and the heights h′ are 50 micrometers, respectively.

Referring to FIGS. 2A to 2C, the first target molecule 1 may be smaller than the second target molecule 5. In one embodiment, for example, the size of the first target molecule 1 may be from about 0.01 micrometer to about 1 micrometer, and the size of the second target molecule 5 may be from about 4 micrometers to about 6 micrometers. When the first and second target molecules 1 and 5 pass through the second channel portions of the comparison example, the first and second target molecules 1 and 5 may be affected by the inertial lift force and the Dean drag force. As illustrated with the dashed lines, the Dean flow may form vortices from a center of the second channel portion in upward and downward directions. The arrows toward the second sidewall S₂ illustrate the strength and direction of the Dean drag force, and the arrows toward a ceiling R illustrate the strength and direction of the inertial lift force.

When a ratio h′/w′ of the height h′ to the width w′ of the second channel portion is 1, the first and second target molecules 1 and 5 are affected more by the Dean drag force than by the inertial lift force. Therefore, the more the first and second target molecules 1 and 5 move toward the fluid outlet portion, the more the first and second target molecules 1 and 5 move toward the second sidewall S₂.

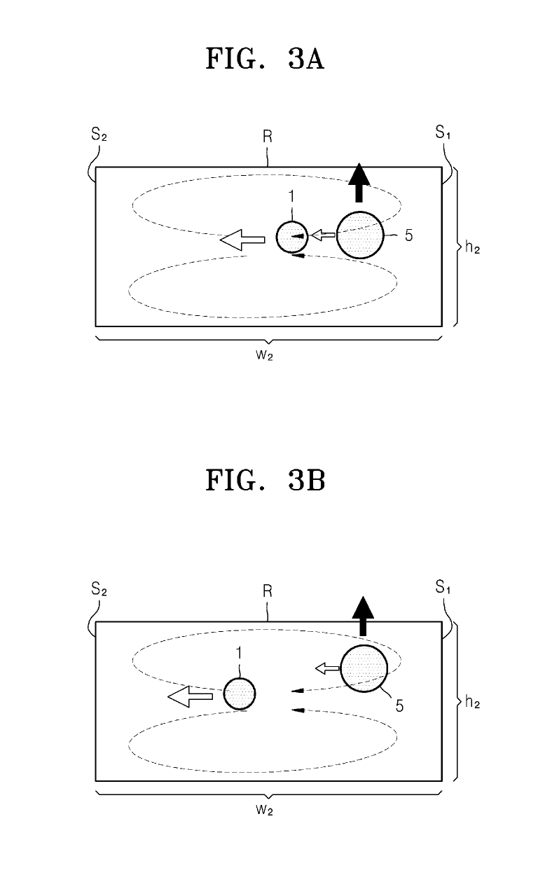

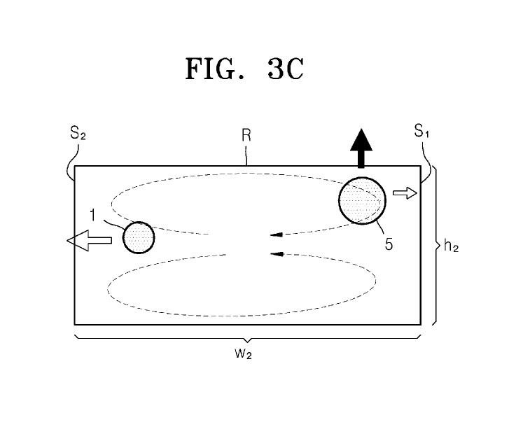

FIGS. 3A to 3C are cross-sectional views illustrating second channel portions of an apparatus according to an embodiment. FIG. 3A illustrates the second channel portion closest to the fluid inlet portion, and FIG. 3C illustrates the second channel portion farthest from the fluid inlet portion. FIG. 3B illustrates the second channel portion disposed between the second channel portions of FIGS. 3A and 3C. The width w₂ of the second channel portion of the embodiment may be larger than the height h₂ of the second channel portion. Here, the width w₂ is about 50 micrometers, and the height h₂ is about 20 micrometers.

Referring to FIGS. 3A to 3C, the first target molecule 1 may be smaller than the second target molecule 5. In one embodiment, for example, the size of the first target molecule 1 may be from about 0.01 micrometer to about 1 micrometer, and the size of the second target molecule 5 may be from about 4 micrometers to about 6 micrometers. When the first and second target molecules 1 and 5 pass through the second channel portions of the illustrated embodiment, the first and second target molecules 1 and 5 may be affected by the inertial lift force and the Dean drag force. The arrows toward the second sidewall S₂ illustrate the strength and direction of the Dean drag force, and the arrows toward the ceiling R illustrates the strength and direction of the inertial lift force.

When a ratio h₂/w₂ of the height h₂ to the width w₂ of the second channel portion is below 1, a shear flow rate at the center of the second channel portion increases. Therefore, the second target molecule 5, which is larger than the first target molecule 1, is relatively affected more by the inertial lift force than by the Dean drag force. Also, the second target molecule 5 is more affected by the inertial lift force than the first target molecule 1 is. With time, as the fluid passes through the second channel portions, the second target molecule 5 moves toward the ceiling R and the first sidewall S₁ of the second channel portion of the illustrated embodiment. The lower the height h₂ of the second channel portion, the faster the shear flow rate and the larger the inertial lift force affecting the target molecule.

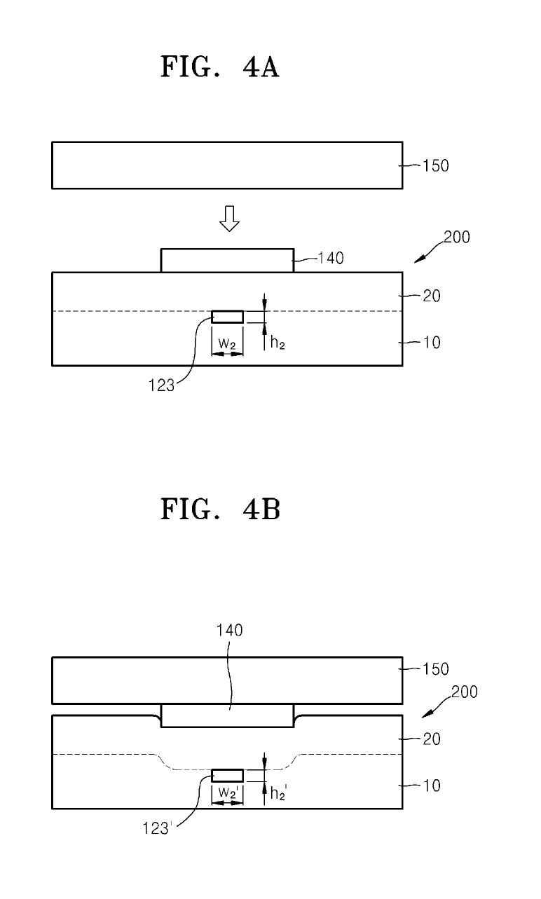

FIG. 4A is a cross-sectional view illustrating a state when no pressure is applied to a fluid channel portion of an apparatus 200 according to an embodiment, and FIG. 4B is a cross-sectional view illustrating a state when a pressure is applied to the fluid channel portion of FIG. 4A.

Referring to FIG. 4A, the apparatus 200 may further include an upper substrate 20 on the substrate 10, and a pressure member 150 configured to apply a pressure to the fluid channel portion. FIG. 4A illustrates a state when no pressure is applied to the fluid channel portion by the pressure member 150.

The upper substrate 20 may include an elastomer such as rubber and a silicone resin. When pressure is applied from outside of the apparatus 200, the substrate 10 and the upper substrate 20 may be deformed elastically. The height of the fluid channel portion may vary depending on the pressure to the fluid channel portion. The fluid inlet portion, the fluid channel portion, and the fluid outlet portion may be on the surface of the substrate 10. The upper substrate 20 may be on the substrate 10 and cover (e.g., overlap) the fluid inlet portion, the fluid channel portion, and the fluid outlet portion.

The pressure member 150 may apply the pressure to the fluid channel portion to change the height of the fluid channel portion. The pressure member 150 may be on the upper substrate 20 to apply the pressure to the fluid channel portion. The apparatus 200 may change the height of the fluid channel portion to control the size for the target molecules to be separated. The pressure member 150 may be a weight having a predetermined mass. Also, the pressure member 150 may include at least one weight. The pressure applied to the fluid channel portion may be adjusted depending on the number of such weights. In one embodiment, for example, the pressure member 150 may include a weight applying about 96 kilopascals (kPa), about 194 kPa, or about 293 kPa of pressure.

FIG. 4A illustrates a second channel portion 123 on the substrate 10, where the width w₂ of the second channel portion 123 may be larger than the height h₂ of the second channel portion 123. When the pressure member 150 is contacted with and then separated from the upper substrate 20, the height of the fluid channel portion may be elastically deformed and then returned to the height the fluid channel portion has when no pressure is applied, respectively.

An auxiliary substrate 140 may be on the upper substrate 20, and between the apparatus 200 and the pressure member 150. The auxiliary substrate 140 may be on an area of the upper substrate 20 corresponding to the fluid channel portion. The auxiliary substrate 140 may concentrate the pressure applied by the pressure member 150 directly on the fluid channel portion. The auxiliary substrate 140 may include a material which may deliver the pressure applied by the pressure member 150 to the fluid channel portion. In one embodiment, for example, the auxiliary substrate 140 may include an acryl, a plastic or glass.

A user of the apparatus 200 may personally apply a force directly to the auxiliary substrate 140 so that the upper substrate 20 is deformed to a predetermined extent by the auxiliary substrate 140. A deformed position of the auxiliary substrate 140 may be held then so that the reduced height of the fluid channel portion decreases and is maintained. Also, when the force to the auxiliary substrate 140 is removed, the height of the fluid channel portion returns to the original distance.

A plurality of the pressure members 150 may be on the upper substrate 20. The pressure members 150 may be parallel on areas corresponding to the first and second channel portions 121 and 123. Individual pressure members 150 may apply different amounts of pressure to the first and second channel portions 121 and 123 to make the heights of the first and second channel portions 121 and 123 different, respectively.

FIG. 4B illustrates a state when the pressure is applied to a fluid channel portion of the apparatus 200 by the pressure member 150.

The substrate 10 and the upper substrate 20 are deformed by the pressure applied by the pressure member 150. In this case, the height of the fluid channel portion is also changed. The pressure applied by the pressure member 150 concentrates on the second channel portion 123 due to the auxiliary substrate 140. The deformed height h₂′ of the second channel portion 123′ after the pressure is applied is lower than the original height h₂ before the pressure is applied. The difference in the widths w₂ and w₂′ of the second channel portions 123 and 123′ before and after the pressure is applied is negligible. When the height h₂′ of the second channel portion 123′ is lowered, the cut-off size for the target molecules may become smaller. In other words, the lower the height h₂′ of the second channel portion 123′, the smaller the target molecules moving toward a sidewall S₁ of the fluid channel portion that can be separated. That is, the same apparatus 200 includes a height that is adjustable according to the desired size of target molecules to be accommodated and separated by the apparatus 200.

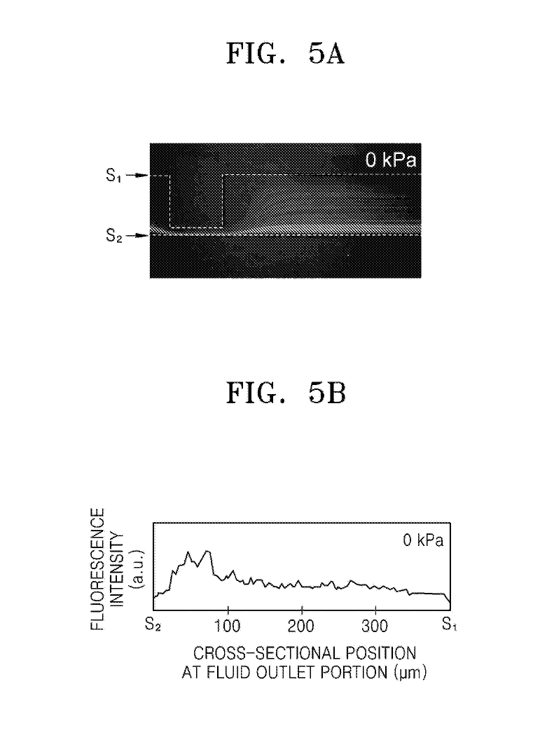

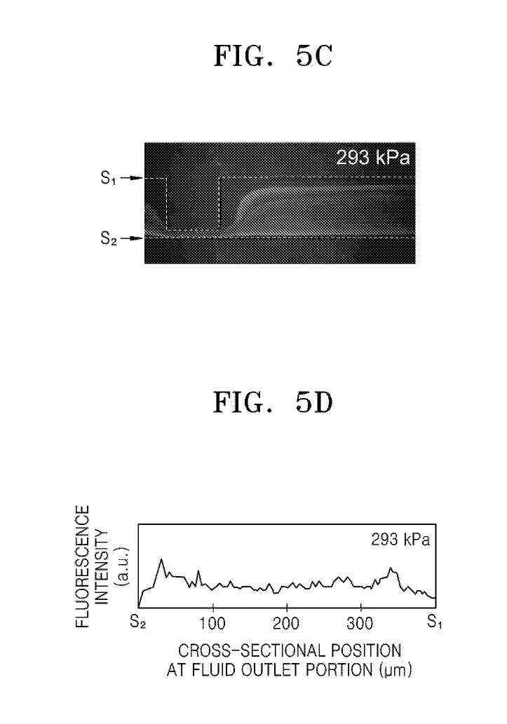

FIGS. 5A through 5D are graphs illustrating how red blood cells contained in the blood are separated by an apparatus according to an embodiment depending on the height of a fluid channel portion. The width W₁ of the first channel portion 121 of the apparatus in an experiment herein is about 350 micrometers, and the width W₂ of a second channel portion 123 of the apparatus is about 50 micrometers. First and second channel portions 121 and 123 alternate with each other six times, and the distance d between leading edges of adjacent second channel portions 123 is about 700 micrometers. Each of the heights h₁ and h₂ is about 50 micrometers before the pressure is applied, and about 20 micrometers after a predetermined amount of pressure (e.g., 293 kPa) is applied. The blood is injected into a first fluid inlet passage at a flow rate of about 1.2 milliliters per hour (ml/h), and a PBS solution is injected into a second fluid inlet passage at a flow rate of about 12 ml/h. The Reynolds number Re is about 12.5.

FIG. 5A illustrates a result of the apparatus separating the red blood cells when the height h₂ and width W₂ of the second channel portion are identical to each other, and FIG. 5B is a graph illustrating a fluorescence intensity in arbitrary units (a.u.) of the separated red blood cells detected at a cross-sectional position in micrometers (μm) of the fluid outlet portion. The blood containing the red blood cells and the blood plasmas are injected into the fluid inlet portion. The red blood cells are more affected by the inertial lift force since the red blood cells are larger than particles in the blood plasmas. However, when the ratio of the height h₂ to width w₂ of the second channel portion is 1, the red bloods cells and the blood plasmas are affected relatively more by the Dean drag force than by the inertial lift force. Therefore, the red blood cells are separated toward the second sidewall S₂. According to FIG. 5B, the fluorescence intensity of the red blood cells is high near the second sidewall S₂.

FIG. 5C illustrates how the red blood cells are separated by the apparatus when the width w₂′ is larger than the height h₂′ of the second channel portion because of the pressure applied to the fluid channel portion, and FIG. 5D is a graph illustrating the fluorescence intensity of the separated red blood cells detected at the fluid outlet portion. The blood containing the red blood cells and the blood plasmas are injected into the fluid inlet portion. The red blood cells are more affected by the inertial lift force since the red blood cells are larger than particles in the blood plasmas. Also, since the ratio of the height h₂′ to width w₂′ of the second channel portion is below 1, the red blood cells are relatively more affected by the inertial lift force. Therefore, the red blood cells move toward the first sidewall S₁ and are separated from the blood plasmas. According to FIG. 5D, the fluorescence intensity of the red blood cells near the first sidewall S₁ is relatively higher when compared with the graph of FIG. 5B.

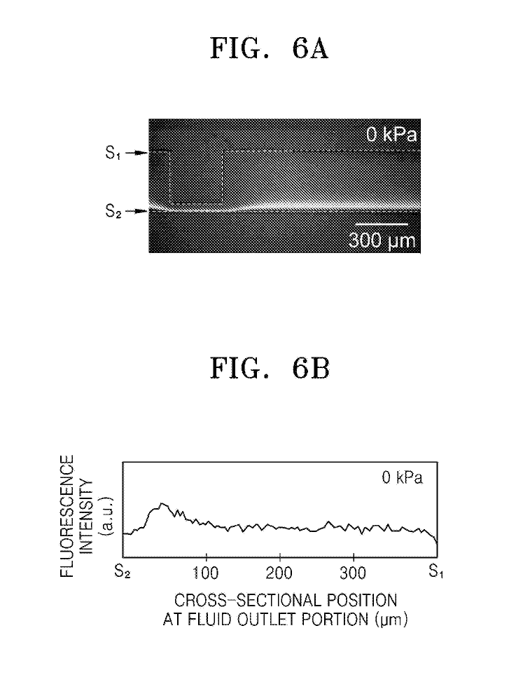

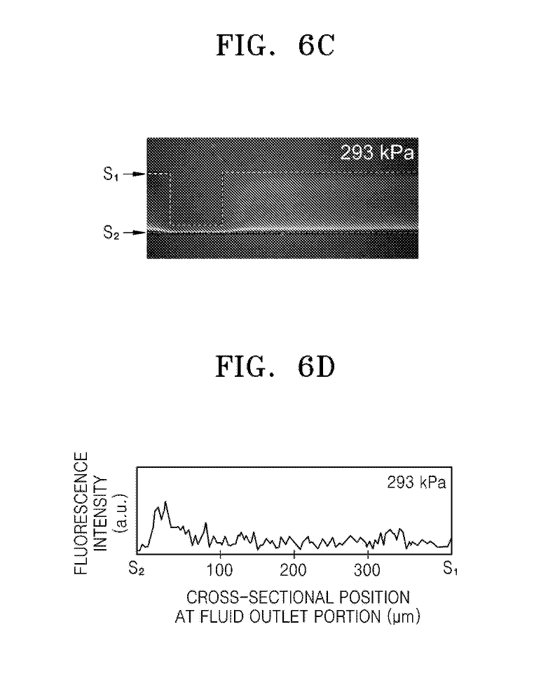

FIGS. 6A through 6D are graphs illustrating how the blood plasmas contained in the blood are separated by an apparatus according to an embodiment depending on the height of a fluid channel portion. The width W₁ of a first channel portion 121 of the apparatus in the experiment herein is about 350 micrometers, and the width W₂ of a second channel portion 123 of the apparatus is about 50 micrometers. The first and second channel portions 121 and 123 alternate with each other six times, and the distance d between leading edges of adjacent second channel portions 123 is about 700 micrometers. Each of the heights h₁ and h₂ is about 50 micrometers before the pressure is applied, and about 20 micrometers after the predetermined amount of pressure (e.g., 293 kPa) is applied. The blood is injected into a first fluid inlet passage at a flow rate of about 1.2 ml/h, and a PBS solution is injected into a second fluid inlet passage at a flow rate of about 12 ml/h. The Reynolds number Re is about 12.5.

FIG. 6A illustrates how the blood plasmas are separated by the apparatus when the height h₂ and width W₂ of the second channel portion are identical to each other, and FIG. 6B is a graph illustrating a fluorescence intensity in arbitrary units (a.u.) of the separated blood plasmas detected at a cross-sectional position in micrometers (μm) of the fluid outlet portion. The blood containing the red blood cells and the blood plasmas are injected into the fluid inlet portion. The particles contained in the blood plasmas are less affected by the inertial lift force since the particles are smaller than the red blood cells. Also, when the ratio of the height h₂ to width w₂ of the second channel portion is 1, the red bloods cells and the blood plasmas are affected relatively more by the Dean drag force than by the inertial lift force. Therefore, the blood plasmas are separated toward the second sidewall S₂. According to FIG. 6B, the fluorescence intensity of the blood plasmas is high near the second sidewall S₂.

FIG. 6C illustrates how the blood plasmas are separated by the apparatus when the width w₂′ is larger than the height h₂′ of the second channel portion because of the pressure applied to the fluid channel portion, and FIG. 6D is a graph illustrating the fluorescence intensity of the separated blood plasmas detected at the fluid outlet portion. The blood containing the red blood cells and the blood plasmas are injected into the fluid inlet portion. The particles contained in the blood plasmas are relatively less affected by the inertial lift force since the particles are smaller than the red blood cells. Also, although the ratio of the height h₂′ to width w₂′ of the second channel portion is below 1, the blood plasmas are relatively less affected by the inertial lift force than the red blood cells are. Therefore, the blood plasmas move toward the second sidewall S₂ and are separated. According to FIG. 6D, the fluorescence intensity of the blood plasmas near the second sidewall S₂ is relatively higher when compared with the graph of FIG. 6B.

Referring to FIGS. 5C, 5D, 6C, and 6D, the apparatus for separating target molecules of the embodiment may separate the red blood cells contained in the blood toward the first sidewall S₁, and the blood plasmas toward the second sidewall S₂. Therefore, an apparatus for separating target molecules and a method using the apparatus may separate the target molecules contained in the fluid depending on the sizes of the target molecules.

It should be understood that the embodiments described therein should be considered in a descriptive sense only and not for purposes of limitation. Descriptions of features or aspects within each embodiment should typically be considered as available for other similar features or aspects in other embodiments.

What is claimed is:

1. An apparatus for separating target molecules, comprising:
a substrate including an elastomer;
a fluid inlet portion on the substrate and comprising at least one fluid inlet passage into which a fluid containing at least one type of target molecule is injected;
a fluid channel portion on the substrate, in fluid connection with the fluid inlet portion, and comprising a plurality of protruding portions on a first sidewall thereof, wherein the protruding portions control a flow of the fluid, and a height of the fluid channel portion is variable so that the target molecules are separated based on a size of the target molecules; and
a fluid outlet portion on the substrate, in fluid connection with the fluid channel portion, and comprising at least one fluid outlet passage through which the separated target molecules are discharged.

2. The apparatus according to claim 1, further comprising a pressure member which overlaps the fluid channel portion, wherein the pressure member applies a pressure to the fluid channel portion so that the height of the fluid channel portion varies.

3. The apparatus according to claim 1, wherein
a ratio of the size of the target molecule to the height of the fluid channel portion is from about 0.05 to about 0.3, and
the target molecules are separated toward the first sidewall.

4. The apparatus according to claim 1, wherein
the fluid channel portion further comprises a plurality of first channel portions separated from each other by the plurality of protruding portions, and a plurality of second channel portions which alternate with the plurality of first channel portions,
the plurality of second channel portions having a width smaller than a width of the plurality of first channel portions.

5. The apparatus according to claim 4, wherein the width of the plurality of second channel portions is larger than a height of the plurality of second channel portions.

6. The apparatus according to claim 5, wherein
a ratio of the size of the target molecule to the height of the plurality of second channel portions is from about 0.05 to about 0.3, and
the target molecules are separated toward the first sidewall.

7. A method of separating target molecules, comprising:
injecting a fluid containing at least one type of target molecules into a fluid inlet portion of an apparatus,
separating the target molecules depending on a size of the target molecules by controlling a flow of the fluid with a fluid channel portion of the apparatus, which is in fluid connection with the fluid inlet portion, and
discharging the fluid containing the separated target molecules through a fluid outlet portion of the apparatus, which is in fluid connection with the fluid channel,
wherein the apparatus comprises:
a substrate including an elastomer,
the fluid inlet portion on the substrate and comprising at least one fluid inlet passage,
the fluid channel portion on the substrate, comprising a plurality of protruding portions on a first sidewall thereof and having a variable height, and
the fluid outlet portion on the substrate and comprising at least one fluid outlet passage.

8. The method according to claim 7, further comprising:
applying a pressure to the fluid channel portion so that the height of the fluid channel portion varies.

9. The method according to claim 7, wherein the height of the fluid channel portion varies so that a ratio of the size of the separated target molecule to the height of the fluid channel portion is from about 0.05 to about 0.3.

10. The method according to claim 7, wherein
the fluid channel portion further comprises a plurality of first channel portions separated from each other by the plurality of protruding portions, and a plurality of second channel portions which alternate with the plurality of first channel portions,
the plurality of second channel portions having a width smaller than a width of the plurality of first channel portions.

11. The method according to claim 10, wherein the width of the plurality of second channel portions is larger than a height of the plurality of second channel portions.

12. The method according to claim 11, wherein the height of the plurality of second channel portions varies so that a ratio of the size of the separated target molecule to the height of the plurality of second channel portions is from about 0.05 to about 0.3.

13. An apparatus for separating target molecules, comprising:
a fluid inlet passage into which a fluid containing at least one type of target molecule is injected;
a fluid outlet passage through which separated target molecules are discharged and in fluid connection with the fluid channel; and
a fluid channel between the fluid inlet passage and the fluid outlet passage, through which the fluid flows in a flow direction and in fluid connection with the fluid inlet passage and the fluid outlet passage, the fluid channel comprising:
a first channel portion having a first width perpendicular to the flow direction,
a second channel portion having a second width perpendicular to the flow direction and smaller than the first width, and
a plurality of protruding portions which are extended from a sidewall of the fluid channel and define the first and second channel portions; and
wherein
a height of the second channel portion is variable and is less than the second width.
